SEQUENCE LISTING (2 sequences)

SEQ ID NO: 1 - protein, 214 aa, Artificial Sequence
  Adalimumab light-chain sequence
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 224 aa, Artificial Sequence
  Adalimumab heavy-chain sequence
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

(19) United States
(12) Patent Application Publication
(10) Pub. No.: US 2017/0121402 A1
(43) Pub. Date: May 4, 2017

(54) ORAL COMPOSITION OF ANTI-TNF ALPHA ANTIBODIES

(71) Applicant: Laboratoire Français du Fractionnement et des Biotechnologies, Les Ulis (FR)

(72) Inventor: Abdessatar Sami Chtourou, Elancourt (FR)

(73) Assignee: Laboratoire Français du Fractionnement et des Biotechnologies, Les Ulis (FR)

(21) Appl. No.: 15/319,178

(22) PCT Filed: Jun. 18, 2015
(86) PCT No.: PCT/FR15/51616

§ 371 Date: Dec. 15, 2016

(30) Foreign Application Priority Data
Jun. 18, 2014 (FR) 1455582

(51) Int. Cl. C07K 16/24; A61K 39/395; A61K 31/20

(52) U.S. Cl. CPC C07K 16/241; A61K 31/20; A61K 39/3955; C07K 2317/12; A61K 2039/542

(57) ABSTRACT

The present invention relates to a pharmaceutical composition for oral delivery, including an anti tumour necrosis factor alpha (TNFα) antibody, preferably a monoclonal antibody produced in the milk of a transgenic non-human animal, and which can be combined advantageously with caprylic acid, and is preferably in a form that is suitable for targeted release of the antibody in the intestine.

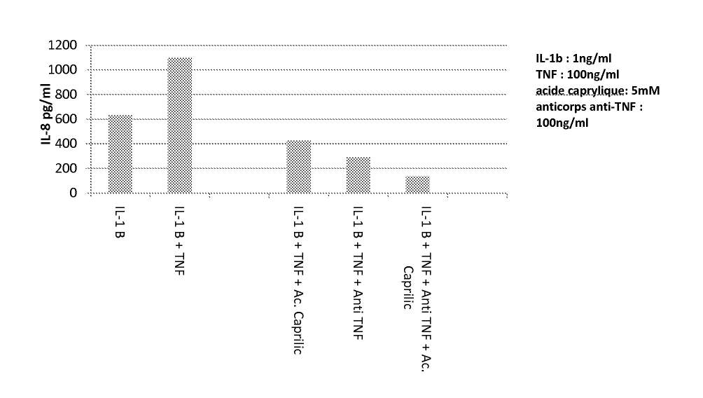

The present invention relates to the oral administration of an anti-tumor necrosis factor alpha (TNFα) antibody. The therapeutic applications concerned notably include the treatment of inflammatory bowel diseases, such as Crohn's disease.

### TECHNOLOGICAL BACKGROUND OF THE INVENTION

TNFα is a proinflammatory cytokine which is secreted by and interacts with cells of the immune system. TNFα has been shown to be involved in many human diseases, notably chronic inflammatory diseases like rheumatoid arthritis, Crohn's disease, ulcerative colitis and multiple sclerosis. Several anti-TNFα antibodies are currently under development. Two antibodies have already been marketed: infliximab (Remicade®) and adalimumab (Humira®), in forms for subcutaneous or intravenous injection.

Several studies have nevertheless reported that anti-TNFα antibody therapy, in particular by systemic route, can have undesirable side effects, notably the occurrence of bacterial infections such as tuberculosis (Jarequi-Amezaga et al., Journal of Crohn's and colitis, 2013, 7(3):208-212), or infections by Listeria (Abreu et al., Journal of Crohn's and colitis, 2013, 7(2):175-182) or by Candida (Huang et al., Journal of pediatric gastroenterology and nutrition, 2013, 56(4):e23-6).

Formulations of anti-TNFα polyclonal antibodies for oral administration are under development. But there still exists a need to improve the efficacy and/or safety of anti-TNFα antibodies administered orally.

### SUMMARY OF THE INVENTION

The inventors now propose a pharmaceutical composition for oral administration, comprising an anti-tumor necrosis factor alpha (TNFα) antibody, preferably a monoclonal antibody produced in the milk of a transgenic non-human animal, and which can be combined advantageously with caprylic acid.

Preferably, the composition is in a form suitable for targeted release of the antibody in the intestine.

In a preferred embodiment, the anti-TNFα antibody is adalimumab or has the protein sequence of adalimumab.

The composition is useful notably in the treatment of inflammatory disease, preferably inflammatory bowel disease, more preferably Crohn's disease.

### DETAILED DESCRIPTION OF THE INVENTION

Definitions:

The term “treatment” or “treat” refers to an improvement in or the prophylaxis or the reversal of a disease or a disorder, or at least a symptom which can be distinguished therefrom, or an improvement in or the prophylaxis or reversal of at least one measurable physical parameter associated with the disease or the disorder being treated, which is not necessarily distinguishable in or by the treated subject. The term “treatment” or “treat” further includes the inhibition or slowing down of the progression of a disease or a disorder, physically, for example, the stabilization of a distinguishable symptom, physiologically, for example, the stabilization of a physical parameter, or both.

Within the meaning of the present invention, by “patient” is meant any mammal, and more particularly human beings, men or women, irrespective of age.

The term “inflammatory disease” includes any disease having an inflammatory component, preferably inflammatory bowel diseases, in particular chronic inflammatory bowel diseases, such as Crohn's disease and ulcerative colitis. Also included are idiopathic juvenile arthritis, rheumatoid arthritis, psoriasis, psoriatic rheumatism and ankylosing spondylitis.

The terms “combination” and “co-administration” refer to the administration of anti-TNFα antibody and caprylic acid in the form of a single formulation or as two separate formulations. The administration may be simultaneous or sequential, in any order. The two agents may be administered simultaneously or successively.

Anti-TNFα Antibody:

By “anti-TNFα antibody” is meant any antibody specifically binding to human TNFα. Preferably, the antibody dissociates from human TNFα according to the following constants: a K_{d} lower than 1×10⁻⁸ M (preferably lower than 1×10⁻⁹ M, preferably lower than 1×10⁻¹⁰ M, preferably lower than 1×10⁻¹¹ M) and a K_{off} of 1×10⁻³ s⁻¹ or lower, both determined by a surface plasmon resonance test. Preferably the antibody is neutralizing, in particular it neutralizes the biological function of TNFα by blocking the interaction thereof with TNF p55 and p75 receptors located on the cell surface. The neutralizing capacity of the antibody can be tested by a standard test, for example the antibody neutralizes the cytotoxicity of human TNFα in a standard L929 in vitro test, with an IC₅₀ of 1×10⁻⁷ M or lower, preferably lower than 1×10⁻⁸ M, 1×10⁻⁹ M or 1×10⁻¹⁰ M.

Preferably it is a monoclonal antibody.

The antibody may be an antibody from a mammal such as a mouse, or may be preferably humanized, or entirely human.

In a preferred embodiment, the anti-TNFα antibody is a human monoclonal antibody.

The anti-TNFα antibody preferentially used is adalimumab or has the protein sequence of adalimumab and namely comprises the light-chain sequence SEQ ID NO: 1 and the heavy-chain sequence SEQ ID NO: 2.

| SEQ ID NO: 1 | |
|---|---|
| 1 | DIQMTQSPSS LSASVGDRVT ITCRASQGIR NYLAWYQQKP GKAPKLLIYA ASTLQSGVPS |
| 61 | RFSGSGSGTD FTLTISSLQP EDVATYYCQR YNRAPYTFGQ GTKVEIKRTV AAPSVFIFPP |
| 121 | SDEQLKSGTA SVVCLLNNFY PREAKVQWKV DNALQSGNSQ ESVTEQDSKD STYSLSSTLT |
| 181 | LSKADYEKHK VYACEVTHQG LSSPVTKSFN RGEC |
| SEQ ID NO: 2 | |
| 1 | EVQLVESGGG LVQPGRSLRL SCAASGFTFD DYAMHWVRQA PGKGLEWVSA ITWNSGHIDY |
| 61 | ADSVEGRFTI SRDNAKNSLY LQMNSLRAED TAVYYCAKVS YLSTASSLDY WGQGTLVTVS |
| 121 | SASTKGPSVF PLAPSSKSTS GGTAALGCLV KDYFPEPVTV SWNSGALTSG VHTFPAVLQS |
| 181 | SGLYSLSSVV TVPSSSLGTQ TYICNVNHKP SNTKVDKKVE PKSC |

Adalimumab is an immunoglobulin G (IgG) consisting of two kappa light chains and two IgG1 heavy chains.

The antibody may also be infliximab or golimumab, for example.

The antibody used in the invention may be produced by any technique known to the person skilled in the art; preferably it is a recombinant antibody.

In a particular embodiment, the antibody may thus be produced by recombination in a host cell, transformed with one or more vectors which allow the expression and/or the secretion of the nucleotide sequences coding for the heavy chain and/or the light chain of the antibody. The vector usually comprises a promoter, translation initiation and termination signals, as well as suitable transcription regulation regions. It is maintained in a stable manner in the host cell and may optionally have specific signals that specify the secretion of the translated protein. These various elements are selected and optimized by the person skilled in the art according to the host cell used. Such vectors are prepared by methods commonly used by the person skilled in the art, and the resulting clones may be introduced into a suitable host by standard methods, such as lipofection, electroporation, the use of polycationic agents, heat shock or chemical methods. The host cell may be selected from prokaryotic or eukaryotic systems, for example bacterial cells but also yeast cells or animal cells, in particular mammalian cells. The preferred mammalian cells for the production of the monoclonal antibody are the rat line YB2/0, the hamster line CHO, in particular the lines CHO dhfr- and CHO Lec13, PER.C6TM (Crucell), 293, K562, NS0, SP2/0, BHK or COS. It is also possible to use insect cells.

Another mode of production is the expression of the recombinant antibody in transgenic organisms, for example in plants (Ayala M, Gavilondo J, Rodríguez M, Fuentes A, Enríquez G, Pérez L, Cremata J, Pujol M. Production of plantibodies in Nicotiana plants. Methods Mol Biol. 2009; 483:103-34) or particularly in the milk of transgenic animals such as rabbit, goat or pig (Pollock, D. P., J. P. Kutzko, E. Birck-Wilson, J. L. Williams, Y. Echelard and H. M. Meade. (1999). Transgenic milk as a method for the production of recombinant antibodies. Journal of Immunological Methods. 231: 147-157).

According to a preferred embodiment, the antibody is produced in the milk of non-human transgenic mammals, genetically modified to produce this glycoprotein. The mammal may be for example a goat, a ewe, a female bison, buffalo, camel, llama, mouse or rat, or a cow, sow, doe rabbit or mare.

Preferably, it is transgenic goat's milk.

Secretion of the antibody by the mammary glands, allowing its presence in the milk of the transgenic mammal, involves tissue-dependent control of the expression of the antibody. Such methods of control are well-known to the person skilled in the art. Expression is controlled by means of sequences allowing expression of the glycoprotein in a particular tissue of the animal. They are in particular promotor sequences of the “WAP”, “β-casein”, “β-lactoglobulin” type and possibly sequences of the peptide signal type. Preferably, the antibody is produced in the mammary glands of a transgenic goat, using an expression vector comprising the sequence of the two chains, under the control of a 5′ β-casein promoter. A process for extracting proteins of interest from the milk of transgenic animals is described in the patent EP 0 264 166.

Advantageously, more than 4 grams of antibody per liter of milk is produced, advantageously more than 5, 10, 15, 20, 25, 30, 35 grams per liter, advantageously up to 70 grams per liter.

Advantageously, the antibody produced by animal transgenesis, in particular in the mammary glands of a transgenic goat, is in the form of a population of anti-TNFα antibodies which have glycosylation with a high level of galactosylation, for example greater than 60%, preferably greater than 70%, more preferably of at least 80%.

According to yet another particular aspect, the level of fucosylation of all the antibodies of the population is at least 50%, and in particular at least 60%.

According to another particular aspect, the population comprises antibodies which comprise mono-galactosylated N-glycans.

According to another particular aspect, the population comprises antibodies which comprise bi-galactosylated N-glycans.

According to another particular aspect, the ratio of the level of galactosylation of the antibodies of the population to the level of fucosylation of the antibodies of the population is 1.0 to 1.4.

According to another particular aspect, at least 35% of the antibodies in the population comprise bi-galactosylated N-glycans and at least 25% of the antibodies in the population comprise mono-galactosylated N-glycans.

According to another particular aspect, the level of sialylation of the antibodies is at least 50%, preferably at least 70%, or at least 90%.

According to yet another particular aspect, the antibodies are fully sialylated.

N-glycan biosynthesis is not regulated by a coding, as is the case with proteins, but is chiefly dependent on the expression and activity of specific glycosyltransferases in a cell. Thus, a glycoprotein, such as an antibody Fc fragment, normally exists as a heterogeneous population of glycoforms which bear different glycans on the same protein backbone.

A population of highly-galactosylated antibodies is an antibody population in which the level of galactosylation of all the antibodies of the population is at least 50%, at least 60%, at least 70%, at least 80%, at least 90%, up to 100% galactosylation.

According to a particular embodiment of the population of highly-galactosylated antibodies, the level of galactosylation of all the antibodies of the population is at least 60%.

The level of galactosylation may be determined with the following formula:

$$\frac{{\sum }_{i = 1}^{n} ( number of Gal ) * ( \% of relative surface area )}{{\sum }_{i = 1}^{n} ( number of A ) * ( \% of relative surface area )} * 100$$

in which:
“n” is the number of N-glycan peaks analyzed on a chromatogram, for example a normal-phase high-performance liquid chromatography (NP HPLC) spectrum,
“number of Gal” is the number of galactoses on the antenna of the glycan corresponding to the peak,
“number of A” is the number of N-acetyl-glucosamine units on the antenna of he glycan form corresponding to the peak (other than the two N-acetyl-glucosamine units of the framework structure common to glycans), and
“% of relative surface area” is the percentage of the area under the corresponding peak.

The level of galactosylation of the antibodies of the antibody population may be determined, for example, by releasing the N-glycans from the antibodies, by resolving the N-glycans on a chromatogram, by identifying the oligosaccharide unit of the N-glycan which corresponds to a specific peak, by determining the intensity of the peak and by applying the data to the above-mentioned formula.

Antibodies which are galactosylated include antibodies which have mono-galactosylated N-glycans and bi-galactosylated N-glycans.

According to a particular aspect of the population of highly-galactosylated antibodies, the population comprises antibodies which comprise mono-galactosylated N-glycans, which may or may not be sialylated. According to a particular aspect of the population of highly-galactosylated antibodies, at least 1%, at least 5%, at least 10%, at least 15%, at least 20%, at least 25%, at least 30%, at least 40%, at least 50%, at least 60%, at least 70%, at least 80%, at least 90%, up to 100% of the N-glycans of the antibodies comprise mono-galactosylated N-glycans. According to yet another particular embodiment of the invention, in the population of highly-galactosylated antibodies, at least 25% of the antibodies comprise mono-galactosylated N-glycans.

According to a particular aspect of the population of highly-galactosylated antibodies, the population comprises antibodies which comprise bi-galactosylated N-glycans, which may or may not be sialylated. According to a particular aspect of the population of highly-galactosylated antibodies, at least 1%, at least 5%, at least 10%, at least 15%, at least 20%, at least 25%, at least 30%, at least 40%, at least 50%, at least 60%, at least 70%, at least 80%, at least 90%, up to 100% of the N-glycans of the antibodies comprise bi-galactosylated N-glycans. According to yet another particular embodiment of the invention, in the population of highly-galactosylated antibodies, at least 35% of the antibodies comprise bi-galactosylated N-glycans.

According to yet another aspect of the population of highly-galactosylated antibodies, the population comprises antibodies which comprise mono-galactosylated N-glycans, which may or may not be sialylated, and antibodies which comprise bi-galactosylated N-glycans, which may or may not be sialylated.

According to a particular aspect of the population of highly-galactosylated antibodies, at least 1%, at least 5%, at least 10%, at least 15%, at least 20%, at least 25%, at least 30%, at least 40%, at least 50%, at least 60%, at least 70%, at least 80%, at least 90%, up to 99% of the N-glycans of the antibodies comprise mono-galactosylated N-glycans, and at least 1%, at least 5%, at least 10%, at least 15%, at least 20%, at least 25%, at least 30%, at least 40%, at least 50%, at least 60%, at least 70%, at least 80%, at least 90%, up to 99% of the N-glycans of the antibodies comprise bi-galactosylated N-glycans.

According to another particular aspect of the population of highly-galactosylated antibodies, at least 25% of the antibodies comprise mono-galactosylated N-glycans, and at least 35% of the antibodies comprise bi-galactosylated N-glycans.

In a preferred embodiment, the anti-TNFα antibody is produced by the mammary epithelial cells of a transgenic mammal for the production in the mammary gland of exogenous, and potentially also transgenic, anti-TNFα antibody for the production of sialyltransferase. The therapeutic antibodies produced thus have a high level of galactosylation, and potentially increased levels of terminal alpha-2,6 sialic acid bonds on their glycan residues linked to the Fc fragment.

In certain embodiments, the antibody has a glycosylation profile with a high mannose content. As used herein, a “glycosylation profile with a high mannose content” refers to an antibody which contains at least one oligomannose or a composition of antibodies in which at least 30% of the antibodies contain at least one oligomannose. In certain embodiments, at least 30%, 40%, 50%, 60%, 70%, 80%, 90% or more of the antibody sugars are non-fucosylated oligomannoses. In other embodiments, less than 50%, 40%, 30%, 20%, 10%, 5% or less of the antibody sugars contain fucose. In another embodiment, the antibodies have a low fucose content and a high oligomannose content. Thus, in other embodiments, at least 30%, 40%, 50%, 60%, 70%, 80% or 90% or more of the antibody sugars are oligomannose and less than 50%, 40%, 30%, 20%, 10% or 5% of the antibody sugars contain fucose. Thus, in another embodiment, at least 30%, 40%, 50%, 60%, 70%, 80% or 90% or more of the antibody sugars are non-fucosylated oligomannose and less than 50%, 40%, 30%, 20%, 10% or 5% of the antibody sugars contain fucose.

According to a particular aspect, the mannose-containing oligosaccharides range from Man5 to Man9, with the number indicating the number of mannose residues. For example, the mannose-containing oligosaccharides may include Man5, Man6, Man7, Man8 and Man9. In certain embodiments, the antibody, such as transgenically-produced adalimumab, has a high Man6 content. In certain embodiments, the major sugar is Man5. In certain embodiments, at least 10%, 15%, or more of the sugars are Man5. Advantageously, at least 20% of the sugars are Man5. In other embodiments, the major sugar is Man6. In certain embodiments, at least 10%, 15%, or more of the sugars of the transgenically-produced antibody are Man6. Advantageously, at least 20% of the sugars are Man6. In other embodiments, the major sugar is Man7. In certain embodiments, at least 10%, 15% or more of the sugars are Man7. Advantageously, at least 20% of the sugars are Man7.

Particularly preferred are antibodies which have a profile with a high level of galactose or mannose, as described above, as they have a high affinity for the FcγRIIIa (CD16) receptor. By high affinity is meant an affinity at least equal to 2×10⁶ M⁻¹, preferably at least equal to 2×10⁷ M ⁻¹, 2×10⁸ M⁻¹ or 2×10⁹ M⁻¹, as determined by Scatchard analysis or BlAcore technology (Label-free surface plasmon resonance based technology). This receptor is found on many immune cells, including natural killer cells, macrophages, neutrophils and mast cells. This affinity for CD16 allows an improvement in complement-dependent cytotoxicity (CDC) or antibody-dependent cell-mediated cytotoxicity (ADCC) activities or in target cell phagocytosis phenomena, compared to non-highly-galactosylated or non-highly-mannosylated antibodies. In certain embodiments, the populations of anti-TNFα antibodies produced in mammary gland epithelial cells are superior, in terms of binding to soluble TNFα, to antibodies produced in cells which are not mammary gland epithelial cells. In certain embodiments, the populations of anti-TNFα antibodies produced in mammary gland cells are superior, in terms of binding to transmembrane TNFα, to antibodies produced in cells which are not mammary gland epithelial cells. Tests for determining the level of binding to soluble TNFα or to transmembrane TNFα are well established (see for example Horiuchi et al., Rheumatology et al. 49:1215).

Combination with Caprylic Acid:

In a preferred embodiment, the anti-TNFα antibody, which is preferably a monoclonal antibody produced in the milk of a transgenic non-human animal, is administered in combination with caprylic acid.

Caprylic acid, also called octanoic acid, is a saturated straightchain fatty acid comprising eight carbon atoms.

Caprylic acid may be used in acid form, or in caprylate salt form. Any pharmaceutically acceptable salt may be envisaged, for example sodium or potassium caprylate.

According to the invention, but without wishing to be bound to a particular mechanism of action, caprylic acid has as an effect of providing an antiseptic (notably antibacterial, antifungal and antiviral) activity in situ, and may advantageously prevent the risks of infections or superinfections subsequent to neutralization of TNFα in the intestinal lumen. The combination of anti-TNF antibody and caprylic acid also has an improved anti-inflammatory effect. Caprylic acid also makes it possible to improve the efficacy of the antibody by protecting it against a too-rapid degradation in the gastrointestinal tract, which ultimately makes it possible to further strengthen the anti-inflammatory effect of the composition.

The invention thus also has as an object a pharmaceutical composition for oral administration, comprising an anti-TNFα antibody and caprylic acid or a caprylate salt.

Another object of the invention is directed at an anti-TNFα antibody for use in the treatment of inflammatory disease by oral administration, in combination with oral administration of caprylic acid (or a caprylate salt).

The antibody and the caprylic acid (or a caprylate salt) are intended for separate, simultaneous or sequential administration.

Another object of the invention is a kit comprising, within the same package:
A first container containing a pharmaceutical composition for oral administration, comprising an anti-TNFα antibody;
A second container containing a pharmaceutical composition for oral administration, comprising caprylic acid (or a caprylate salt).

The anti-TNFα antibody and the caprylic acid (or a caprylate salt) may be combined within the same pharmaceutical composition, or may be used in the forms of separate compositions, administrable simultaneously or sequentially. In particular, they may be administered separately, namely either concomitantly or independently, for example separated by time. Caprylic acid (or a caprylate salt) may be used in the invention in the form of a pharmaceutical composition or a dietary supplement-type composition.

Therapeutic Formulations and Applications:

The compositions of the present invention may appear in any galenic form normally used for oral administration, particularly in the form of tablets, capsules, powder or any solid oral preparation form or any liquid oral preparation form.

In a preferred embodiment, the composition appears in a galenic form suitable for targeted release in the intestine. By “intestine” is meant herein all parts of the intestine, particularly the colon. Such compositions are particularly useful in the treatment of inflammatory bowel diseases because they allow local action at the site of infection (notably the small intestine or the colon). Moreover, they limit passage of the antibodies into the bloodstream, limiting the side effects associated with anti-TNFα antibodies.

Several strategies exist for preparing orally-administered medicines whose active ingredients are released only in the intestine, preferably only in the colon.

Certain strategies comprise covalent binding of the medicine with a substrate or coating with pH-sensitive polymers. It is also possible to use excipients and vehicles which are degraded by bacteria of the colon.

In a particular embodiment, the anti-TNFα antibody may be formulated in solid dosage forms, like tablets or capsules, coated with pH-sensitive polymers. The pH is on the order of 1 to 3 in the stomach but it increases in the small intestine and colon to reach values close to 7. The most commonly used polymers, insoluble at acidic pH but soluble at neutral or alkaline pH, are methacrylic acid derivatives, notably the polymers Eudragit® L and S.

In another embodiment, formulations coated with polymers degradable by microorganisms of the colon (more particularly by bacterial enzymes like azoreductases and glycosidases), for example azo polymers having a high degree of hydrophilicity, may also be used.

Gels and hydrogels may also be used, notably polysaccharide-based hydrogels.

When the anti-TNFα antibody is combined with caprylic acid (or caprylate salt), the dosage form comprising the anti-TNFα antibody may be different from the dosage form comprising the optionally combined caprylic acid.

Preferably, the anti-TNFα antibody is administered in tablet or capsule form. Preferably, the caprylic acid is administered in capsule form.

The compositions of the invention may comprise or be combined with other therapeutic agents, but in a non-preferred manner.

The compositions according to the invention advantageously comprise one or more pharmaceutically acceptable excipients or vehicles. Mention may be made for example of saline, physiological, isotonic and buffer solutions, etc., compatible with pharmaceutical use and known to the person skilled in the art. The compositions may contain one or more agents or vehicles selected from dispersants, solubilizers, stabilizers, preservatives, etc. Agents or vehicles usable in the formulations (liquid and/or injectable and/or solid) are in particular methylcellulose, hydroxymethylcellulose, carboxymethylcellulose, polysorbate 80, mannitol, gelatin, lactose, vegetable oils, acacia, etc. The compositions may optionally be formulated by means of galenic forms or devices providing prolonged and/or delayed release. For this type of formulation, an agent such as cellulose, carbonates or starches are advantageously used.

The administered doses may vary, inter alia, according to the patient's weight and age, and the severity of the disease, assessed by the person skilled in the art.

In a preferred embodiment, the dose of antibody is in the range of about 0.02 mg/kg to about 100 mg/kg of body weight, or about 1 mg to about 8 g per day for a person weighing 55 to 80 kg.

In a more preferred embodiment, the dose of antibody is in the range of about 0.16 mg/kg to about 32 mg/kg of body weight, or about 8.75 mg to about 2.5 g per day. In a more preferred embodiment, the dose of antibody is in the range of about 0.16 mg/kg to about 13 mg/kg of body weight, or about 8.75 mg to about 1 g per day. In a more preferred embodiment, the dose of antibody is in the range of about 0.16 mg/kg to about 6.5 mg/kg of body weight, or about 8.75 mg to about 520 mg. In a more preferred embodiment, the dose of antibody is in the range of about 0.16 mg/kg to about 3.2 mg/kg of body weight, or about 8.75 mg to about 256 mg. In a more preferred embodiment, the dose of antibody is in the range of about 0.16 mg/kg to about 1.3 mg/kg of body weight, or about 8.75 mg to about 104 mg. In a more preferred embodiment, the dose of antibody is in the range of about 0.16 mg/kg to about 0.64 mg/kg of body weight, or about 8.75 mg to about 51 mg.

Generally, advantageous doses are doses of 8 to 200 mg per day, preferably 8 to 35 or 15 to 70 mg per day.

In a preferred embodiment, the dose of caprylic acid (or caprylate salt) is in the range of 0.01 mg/kg to about 500 mg/kg, for example 0.1 mg/kg to 300 mg/kg, or about 0.1 mg to 20 g per day.

The patient may receive for example a daily dose of anti-TNFα antibody (preferably adalimumab) for example between 10 mg and 500 mg, preferably 8 to 200 mg per day, preferably 8 to 35 or 15 to 70 mg per day

The administered dose of caprylic acid (or caprylate salt) may preferably be between 100 mg and 4 g, preferably between 200 mg and 2 g, preferably between 300 mg and 1.5 g, preferably between 750 mg and 1250 mg, and more preferentially between 900 mg and 1100 mg. It may be a daily dose or a dose taken one or more times per week.

In a particular embodiment, the antibody and the caprylic acid (or caprylate salt) may be used in a weight ratio of caprylic acid to antibody at least equal to 1:1, preferably 5:1, preferably 10:1, preferably 15:1, preferably 20:1.

When the two agents are administered sequentially, the dose is administered either the same day, in at least two separate administrations, or else one of the agents is administered the first day and the second the following day, or the day after for example.

The following example and the appended figure illustrate the invention without limiting its scope.

### FIGURE LEGEND

The appended FIG. is a histogram showing the levels of interleukin-8 secretion by Caco-2 cells, subjected to various conditions. Secretion is most strongly inhibited in the presence of a combination of caprylic acid+anti-TNF antibody.

### EXAMPLE

### Anti-Inflammatory Effect on the Caco-2 Line

Materials and Methods:

Caco-2 cells, a colon cancer cell line, are an established cell model of the human intestinal epithelium (Pinto et al., 1983, Biol. Cell, 47, 323-330). These cells secrete interleukin-8 (IL-8), which is one of the major interleukins in the pathogenesis of inflammatory bowel diseases, after activation by interleukin-1 beta (IL-1 beta).

The inventors used the Caco-2 cell line as a model of epithelial cells, and evaluated the effect of compositions according to the invention on interleukin-8 (IL-8) secretion by these cells after activation by interleukin-1 beta (IL-1 beta).

To that end, Caco-2 cells, deposited in 24well plates, were cultured for 14 days until the cells were confluent and in a stable differentiation state. On day 14, the cells were precultured in the presence of caprylic acid, transgenically-produced anti-TNF antibody (adalimumab), and a combination of the two. Twenty-four hours later, the cells were stimulated with 1 ng/ml IL-1 beta for 12 hours. IL-8 levels present in the culture supernatants were measured by ELISA.

Results:

The addition of anti-TNF antibody induces a decrease in IL-8 levels, and thus an anti inflammatory potential. The combination of caprylic acid+anti-TNF antibody appears to be a stronger inhibitor of IL-8 secretion (see the figure), showing a more strongly anti inflammatory effect.

The use of Caco-2 cells differentiated after 14 days of culture, and the addition of IL-1 beta, made it possible to reproduce in vitro conditions similar to intestinal inflammation in vivo. The results observed show that a composition according to the invention has a potential therapeutic benefit in vivo.

1. A pharmaceutical composition suitable for oral administration, comprising a monoclonal anti-tumor necrosis factor alpha (TNFα) antibody produced in the milk of a transgenic non-human animal.

2. The pharmaceutical composition according to claim 1, the composition being in a form suitable for targeted release of the antibody in the intestine.

3. The pharmaceutical composition according to claim 1, wherein the anti-TNFα antibody is adalimumab or has the protein sequence of adalimumab.

4. The pharmaceutical composition according to claim 1, wherein the transgenic non-human animal is a goat.

5. The pharmaceutical composition according to claim 1, further comprising caprylic acid or a caprylate salt.

6. A method for treating an inflammatory disease in a patient, which method comprises administering to said patient a pharmaceutical composition suitable for oral administration, comprising a monoclonal anti-tumor necrosis factor alpha (TNFα) antibody produced in the milk of a transgenic non-human animal.

7. The method according to claim 6, wherein the inflammatory disease is inflammatory bowel disease, such as Crohn’ s disease.

8. The method according to claim 6 wherein the pharmaceutical composition is administered orally at a daily dose of 8 to 200 mg, preferably 8 to 35 or 15 to 70 mg of anti-TNFα antibody.

9. A method for treating an inflammatory disease in a patient, which method comprises orally administering to the patient an anti-TNFα antibody, in combination with preferably oral administration of caprylic acid or a caprylate salt.

10. The method according to claim 9, wherein the inflammatory disease is inflammatory bowel disease, preferably Crohn's disease.

11. The method according to claim 9, wherein said antibody is adalimumab or has the protein sequence of adalimumab.

12. The method according to claim 9, wherein said antibody is a monoclonal antibody produced in the milk of a transgenic non-human animal, preferably a goat.

13. The method according to claim 9, wherein, the antibody and the caprylic acid or the caprylate salt are administered separately, simultaneously or sequentially.

14. The method according to claim 9, wherein said antibody is in a form suitable for targeted release of the antibody in the intestine.

15. Kit comprising, within the same package:
A first container containing a pharmaceutical composition for oral administration, comprising an anti-TNFα antibody, which is preferably a monoclonal antibody produced in the milk of a transgenic non-human animal, preferably a goat;
A second container containing a pharmaceutical composition for oral administration, comprising caprylic acid or a caprylate salt.
